(19) United States
(12) Patent Application Publication
(10) Pub. No.: US 2008/0221614 A1
(43) Pub. Date: Sep. 11, 2008

(54) Method for Closing an Arteriotomy

(75) Inventor: Juan-Pablo Mas, Somerville, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/684,061

(22) Filed: Mar. 9, 2007

(51) Int. Cl. A61B 17/08

(52) U.S. Cl. 606/213

(57) ABSTRACT

A method for closing an arteriotomy includes providing a guiding system and a closure system. The guiding system advanced through a sheath extending through the arteriotomy and into the blood vessel lumen. The guiding system includes a pair of stabilization wire guides, each guide including a retention foot disposed at a distal portion thereof. The retention feet are deployed within the lumen. With the entire sheath outside of the vessel, the closure device is then further advanced along the guiding system, or the sheath is pulled proximally over the closure device, thereby stretching or tearing a distal portion of the sheath. The closure system includes a closure device, such as a stapler including a staple that is actuated to close the arteriotomy. The retention feet are undeployed and the guiding system is removed from the body.

### FIELD OF THE INVENTION

The present disclosure relates to a system and method for closing an opening in a vessel wall after a medical procedure, and in particular, to a system and method for closing a puncture arteriotomy after an intra-luminal procedure such as catheterization.

### BACKGROUND OF THE INVENTION

Catheters/catheterization procedures for diagnosis or treatment of cardiovascular and/or peripheral vascular diseases are well known, and typically involve the Seldinger technique to make insertions through layers of tissue and through a wall of the femoral artery. After a diagnostic or interventional catheterization, the arteriotomy puncture formed by the catheter or introducer sheath must be closed. The puncture opening in the artery typically ranges from 5 French (0.0655 inch, 1.67 mm) such as for a diagnostic angiography procedure to as large as 30 French (0.393 inch, 10.00 mm) for an interventional procedure such as implanting an inferior vena cava (IVC) filter. Traditionally, intense pressure has been applied to the puncture site for at least 30-45 minutes after removal of the catheter. Patients who have had a femoral artery puncture are then required to remain at bed rest, essentially motionless and often with a heavy sandbag placed on their upper legs, for several hours to ensure that the bleeding has stopped. Other approaches include systems and methods for blind delivery, viz., without direct visualization of a thrombotic or collagen plug or slurry, and/or suturing methodologies for sealing the puncture. Other known systems and methods blindly deliver a staple or clip to gather and hold together sides of the arteriotomy.

Despite the advantages of known blind closure systems and methods, some systems may actually enlarge the arteriotomy during the procedure of closing it. It is understood that larger vessel openings have the disadvantage of taking longer to achieve hemostasis than relatively smaller vessel openings. Therefore, it is desirable to deliver a blind closure system through a standard catheter introducer sheath that was already in-situ at the end of a catheterization procedure. Alternatively, what is needed is a system and method for blind closure of a puncture arteriotomy without further enlarging or dialing the opening beyond the size that was created for the diagnostic or interventional catheterization procedure.

### BRIEF SUMMARY OF THE INVENTION

The disclosure provides a system and method for closing an arteriotomy in a wall of a vessel of a body after an intra-luminal procedure is disclosed. Although the medical term “arteriotomy” means an incision or opening into an artery, the term is used more generally in the specification to describe openings in both veins and arteries, as well as openings in other hollow body vessels or organs. The terms “distal” and “proximal” are used in the specification with respect to a position or a direction relative to the treating clinician. “Distal” or “distally” are a position distant from or in a direction away from the clinician. “Proximal” and “proximally” are a position near or in a direction toward the clinician.

In an embodiment of the present method, a sheath is disposed through a wall of a vessel and extends partially within a lumen thereof. A guiding system and a vascular closure system are inserted into the sheath. The guiding system includes a pair of stabilization wire guides, each including a retention foot disposed at a distal portion thereof. The stabilization wire guides are advanced through the sheath and into the lumen. The retention feet are then deployed within the lumen. The guiding system, the closure system, and the sheath are pulled proximally until tactile resistance is felt due to the retention feet abutting the inner surface of the vessel wall, at which time, the entire sheath is outside of the vessel. The closure device is then further advanced along the guiding system, thereby radially stretching or tearing a distal portion of the sheath. The closure system includes a closure device, such as a stapler including a staple that is actuated to close the arteriotomy. The retention feet are undeployed and the guiding system is removed from the body.

In another embodiment of the present method, a sheath is disposed through a wall of a vessel and extends partially within a lumen thereof. A guiding system and a vascular closure system are inserted into the sheath. The guiding system includes a pair of stabilization wire guides, each including a retention foot disposed at a distal portion thereof. The stabilization wire guides are advanced through the sheath and into the lumen. The retention feet are then deployed within the lumen. The guiding system, the closure system, and the sheath are pulled proximally until tactile resistance is felt due to the retention feet abutting the inner surface of the vessel wall, at which time the entire sheath is outside of the vessel. The sheath is then further pulled back over the closure device, thereby radially stretching or tearing a distal portion of the sheath. The closure system is then advanced over the guiding system to a position adjacent the arteriotomy. The closure system includes a closure device, such as a stapler including a staple that is actuated to close the arteriotomy. The retention feet are undeployed and the guiding system is removed from the body.

### BRIEF DESCRIPTION OF DRAWINGS

The foregoing and other features and advantages of the present disclosure will be apparent from the following description of the disclosure as illustrated in the accompanying drawings. The accompanying drawings, which are incorporated herein and form a part of the specification, further serve to explain the principles of the disclosure and to enable a person skilled in the pertinent art to make and use the disclosure. The drawings are not to scale.

FIG. 1 is a longitudinal cross-sectional view of body tissue including a vessel with a sheath disposed partially therein.

FIG. 2 is longitudinal cross-sectional view of all the elements of FIG. 1 with a guiding system inserted through the sheath.

FIG. 3 is longitudinal cross-sectional view of the all the elements of FIG. 2 with retention feet of the guiding system deployed within the vessel.

FIG. 4 is a longitudinal cross-sectional view of all the elements of FIG. 3 with the sheath, closure system, and guiding system moved proximally such that the retention feet contact an inner surface of the vessel wall and the sheath is disposed outside of the vessel.

FIG. 5 is a longitudinal cross-sectional view of all the elements of FIG. 4 with the closure system advanced towards the arteriotomy.

FIG. 6 is a longitudinal cross-sectional view of all the elements of FIG. 5 with the closure system adjacent the arteriotomy and the sheath torn or stretched over the closure system.

FIG. 7 is a longitudinal cross-sectional view of all the elements of FIG. 6 with the closure device actuated and the guiding system undeployed for removal from the vessel and body tissue.

FIG. 8 is a longitudinal cross-sectional view of body tissue including a vessel with a sheath disposed partially therein in accordance with another embodiment of the disclosure.

FIG. 9 is a longitudinal cross-sectional view of all the elements of FIG. 8 with a guiding system inserted through the sheath.

FIG. 10 is a longitudinal cross-sectional view of all the elements of FIG. 9 with retention feet of the guiding system deployed within the vessel.

FIG. 11 is a longitudinal cross-sectional view of all the elements of FIG. 10 with the sheath, closure system, and guiding system moved proximally such that the retention feet contact an inner surface of the vessel wall and the sheath is disposed outside of the vessel.

FIG. 12 is a longitudinal cross-sectional view of all the elements of FIG. 11 with the sheath pulled further proximally over the closure system such that the sheath tears or stretches over the closure system.

FIG. 13 is a longitudinal cross-sectional view of all the elements of FIG. 12 with the closure system further advanced to a position adjacent the arteriotomy.

FIG. 14 is a longitudinal cross-sectional view of all the elements of FIG. 13 with the closure device actuated and the guiding system undeployed and partially removed from the vessel and body tissue.

FIG. 15 is a longitudinal cross-sectional view of all the elements of FIG. 6 except an alternative embodiment of a sheath.

FIG. 16 is a longitudinal cross-sectional view of all the elements of FIG. 10 except an alternative embodiment of a sheath.

FIG. 17 is a longitudinal cross-sectional view of all the elements of FIG. 12 except an alternative embodiment of a sheath.

### DETAILED DESCRIPTION OF THE INVENTION

Specific embodiments of the present disclosure are now described with reference to the figures, where like reference numbers indicate identical or functionally similar elements. The present disclosure is directed to a device and method for closing an arteriotomy after a medical procedure in which a vessel wall was punctured to gain access to the vessel lumen.

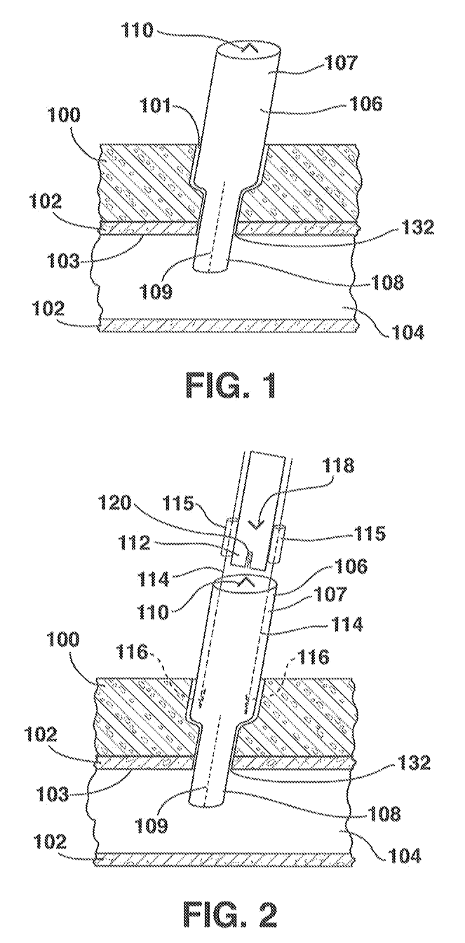

FIG. 1-7 show cross-sectional view of body tissue 100 with a vessel 102 disposed therein. FIG. 1 shows a sheath 106 extending from a proximal portion 107 disposed outside a patient's body to a distal portion 108 disposed partially within a lumen 104 of vessel 102. Sheath 106 maintains access to lumen 104 through a tissue track 101 in tissue 100 and arteriotomy 132 in a wall 103 of vessel 102. Sheath 106 may be used for access to vessel 102 for an intra-luminal procedure, such as balloon angioplasty. Upon completion of such an intra-luminal procedure, sheath 106 may also be used for access to vessel 102 to close arteriotomy 132 in vessel 102.

In the embodiment of sheath 106 shown in FIGS. 1-7, sheath 106 is tapered and/or stepped in diameter such that proximal portion 107 has a larger diameter than distal portion 108. For example, and not by way of limitation, proximal portion 107 may be 8 French (2.67 mm, 0.1048 inches) in size and distal portion 108 may be 6 French (2.00 mm, 0.0786 inches) in size. Sheath 106 further includes a longitudinal perforation 109 in distal portion 108 such that distal portion 108 if tearable, as explained in further detail below.

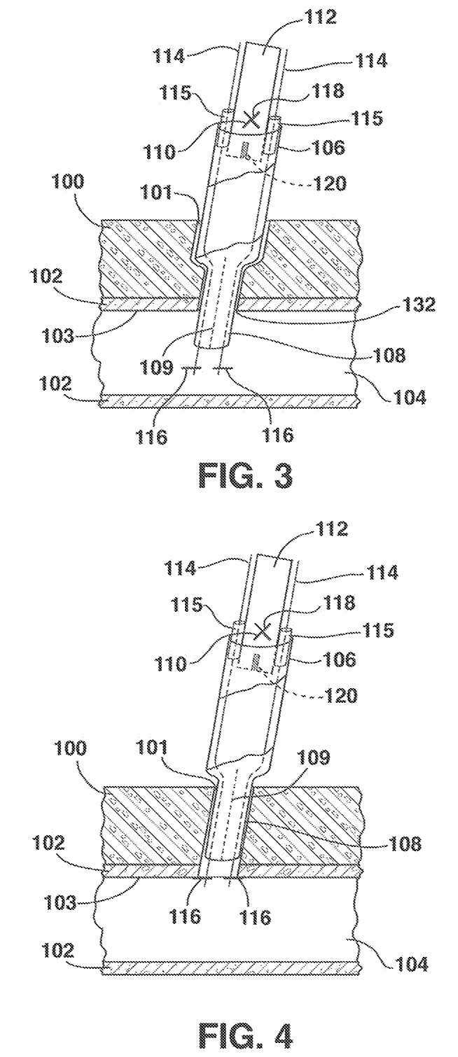

After completion of an intra-luminal procedure and removal of the procedural device, a vascular closure system is used to close arteriotomy 132, as follows. As shown in FIG. 2, a guiding system including a pair of stabilization wire guides 114 is inserted into sheath 106, which may be indwelling at the end of the procedure, or inserted anew, or sheath 106 may replace a sheath that was used during the procedure. The guiding system is advanced through sheath 106 and into lumen 104, as shown in FIGS. 2 and 3. A distal portion of each stabilization wire guide 114 includes a retention foot 116. The guiding system is advanced through sheath 106 and into lumen 104 in an undeployed configuration wherein the retention feet 116 are unexpanded, undeployed or hingedly disposed substantially parallel to stabilization wire guides 114, as shown in FIG. 2, to provide a low cross-sectional profile during insertion.

As shown in FIG. 3, the guiding system is advanced far enough into lumen 104 of vessel 102 to ensure that retention feet 116 are intraluminal prior to their deployment. Numerous methods are known to those of skill in the art to measure or accurately indicate the intraluminal position of portions of a vascular closure device. In one known device, the intraluminal position of the device may be indicated by blood entering the device through an inlet port positioned within the vessel lumen, the blood flowing through a passageway in the device, and the blood finally emitting from the device as a visible “flashback” out of an exit port external to the patient. In an alternative known type of position indicator, the device may have an enlarged physical stop for abutting against the external wall of the blood vessel to provide a tactile indication of the position of the device with respect to the vessel wall. Further, in the embodiment shown in FIGS. 1-7, sheath 106 includes a mating lock 110 in proximal portion 107, and a closure device 112 includes a first mating lock 118. Mating locks 110 and 118 engage in a first locking position shown in FIG. 3.

Once retention feet 116 are positioned intraluminally, the guiding system is transformed into a deployed configuration such that retention feet 116 expand or project laterally from wire guides 114 within lumen 104. Retention feet 116 may be formed and deployed in a various ways as would be known to those of ordinary skill in the art. For example, several retention feet are described in association with FIGS. 49-57 of U.S. Pat. No. 6,767,356 to Kanner et al., the entire disclosure of which is incorporated herein by reference. After retention feet 116 have been deployed such that they are generally perpendicular to stabilization wire guides 114, the entire system, including the guiding system, closure device 112, and sheath 106, is moved proximally until tactile resistance is felt due to retention feet 116 abutting the inner surface of vessel wall 103, as shown in FIG. 4.

The contact of retention feet 116 against the inner surface of vessel wall 103 provides temporary anchoring for stabilization wire guides 114, and also provides an indication of the location, or depth of vessel wall 103 relative to other components of the vascular closure system. For convenience of illustration, FIGS. 2-7 show a pair of stabilization wire guides 114 aligned along the axis of vessel 102. In actual use however, such a pair of stabilization wire guides 114 will typically be aligned transverse to the axis of vessel 102 and positioned at the ends of the slit that naturally forms generally perpendicular to the length of the vessel when arteriotomy 132 is formed in the wall of vessel 102, as described in the above-mentioned Kanner '356 patent. Such transverse alignment of stabilization wire guides 114 within arteriotomy 132 is especially typical when stabilization wire guides 114 are spread apart by closure system 112, as described below.

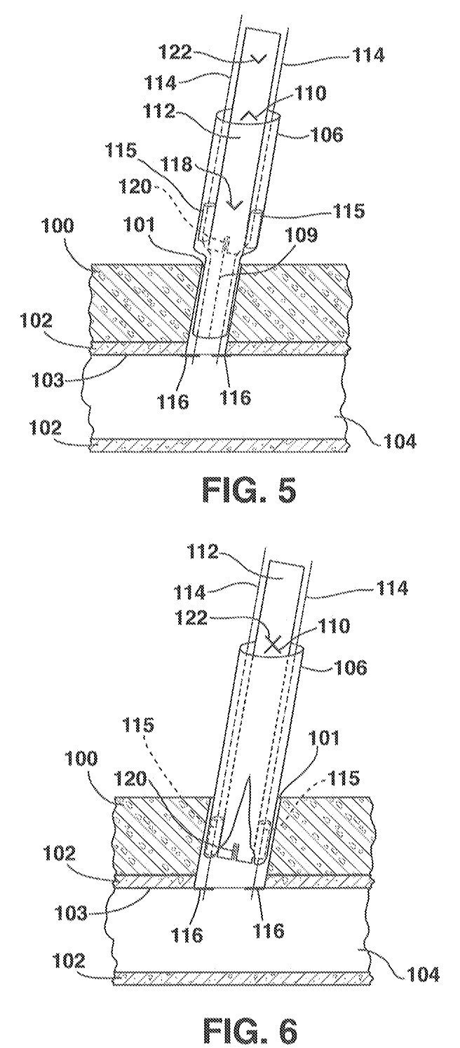

With the guiding system temporarily aligned with arteriotomy 132 by stabilization wire guides 114 and anchored by retention feet 116, closure device 112 is advanced distally along stabilization wire guides 114, as shown in FIGS. 5 and 6. As shown in FIG. 2, two or more relatively short side tubes 115 are coupled parallel to a distal portion of closure device 112 on generally opposing sides of thereof for riding along stabilization wire guides 114. For example, but not as a limitation, closure device 112 of this disclosure may comprise center tube 124 disclosed in U.S. application Ser. No. 11/626,567, filed Jan. 24, 2007, the entirety of which is incorporated by reference herein. As closure device 112 is advanced through sheath 106, distal portion 108 tears along perforation 109, as shown in FIG. 6. Although sheath 106 of FIGS. 1-7 is shown with a tearable distal portion 108 and perforations 109, it would be understood to one of ordinary skill in the art that sheath 106 need not include perforations in order to be tearable. For example, known prior art introducer sheaths are tearable along one or more longitudinal grooves or score lines formed therein. In another example, known prior art introducer sheaths comprise axially oriented polymeric material that will promote a longitudinal tear. Further, instead of being tearable, a sheath 106′, as shown in FIG. 15, may be sufficiently pliant such that it stretches over closure device 112 as closure device 112 is advanced through a distal portion 108′ of sheath 106′.

Stabilization wire guides 114 are also spread apart from each other by closure system 112 as it is advanced through distal portion 108 of sheath 106. With stabilization wire guides 114 positioned at the ends of the arteriotomy slit 132 as described above, spreading stabilization wire guides 114 apart causes the slit to lengthen without enlarging arteriotomy 132, thus drawing the opposite edges of the slit closer towards apposition and enhancing the chances of achieving a successful wound closure.

Closure device 112 may be advanced through sheath 106 far enough such that the closure device distal end may align with, or extend distally from sheath 106, as shown in FIG. 6. Closure device 112 is advanced through sheath 106 and surrounding tissue 100 until closure mechanism 120 carried at the distal end of closure device 112 is suitably positioned adjacent to, or somewhat spaced away from the outer surface of vessel wall 103 in preparation for actuation and/or delivery of closure mechanism 120. The exact positioning of closure mechanism 120 with respect to the outer surface of vessel wall 103 will depend upon the type of closure mechanism being used, as discussed below.

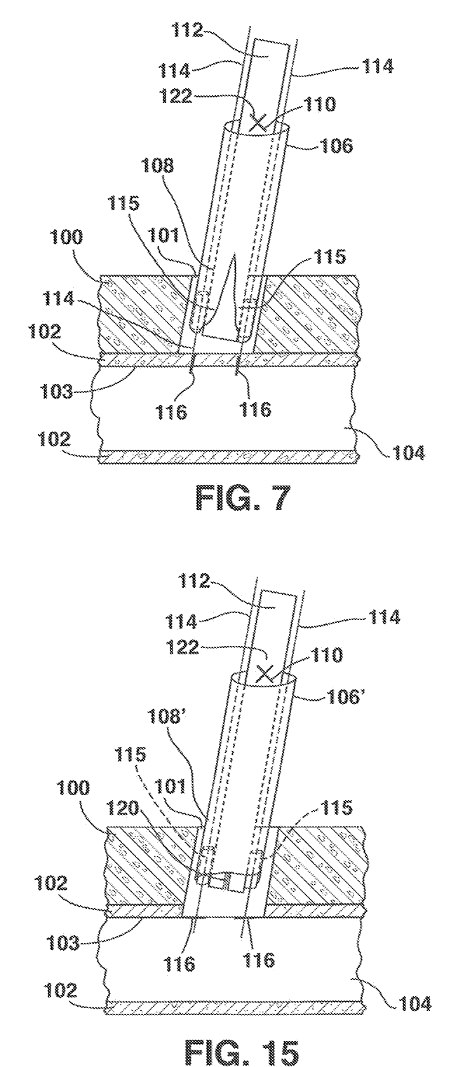

After closure device 112 is advanced through distal portion 108 of sheath 106, closure mechanism 120, such as a staple, is actuated and/or delivered by closure device 112 to close arteriotomy 132. Any conventional vascular puncture closure device can be used, as would be understood by those of ordinary skill in the art. Non-limiting examples of closure devices include implantable mechanisms such as staples, clips, sutures and plugs, or instruments that weld or bond tissue, as by biocompatible adhesive, radio frequency or laser radiation. In particular, the stapler and staples disclosed in U.S. application Ser. No. 11,626,567, incorporated by reference herein above, may be used to close arteriotomy 132. Several methods for releasing closure mechanism 120 from closure device 112 may be used, such as the methods described in the above-referenced U.S. application Ser. No. 11/626,567. Regardless of the closure device or method used, the opposing edges of arteriotomy 132 are brought into apposition surrounding stabilization wire guides 114. After closure mechanism 120 is successfully actuated and/or delivered to close arteriotomy 132, the guiding system is transformed back into the undeployed configuration, such that retention feet 116 are retracted or collapsed into their low cross-sectional profiles, as shown in FIG. 7. Then, the guiding system, closure device 112, and sheath 106, either together or separately, are removed from the patient via tissue track 101.

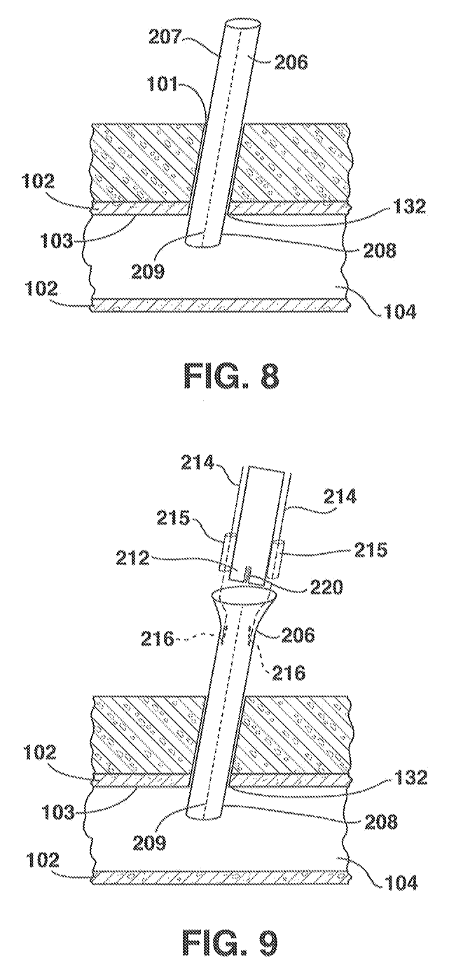

Another embodiment of the present disclosure is illustrated in FIGS. 8-14, which show cross-sectional vies of body tissue 100 with vessel 102 disposed therein. FIG. 8 shows a sheath 206 extending from a proximal portion 207 disposed outside a patient's body to a distal portion 208 disposed partially within lumen 104 of vessel 102. Sheath 206 maintains access to lumen 104 through tissue track 101 in tissue 100 and arteriotomy 132 in wall 103 of vessel 102, similar to sheath 106 shown in FIG. 1. Sheath 206 may be used for access to vessel 102 for an intra-luminal procedure, such as balloon angioplasty. Upon completion of such an intra-luminal procedure, sheath 206 may also be used for access to vessel 102 to close arteriotomy 132.

In contrast to sheath 106 described above, the embodiment of sheath 206 shown in FIGS. 8-14 has a diameter that is not tapered or stepped. Sheath 206 includes a perforation 209 that runs along proximal portion 207 and distal portion 208 such that sheath 206 is tearable, as explained in further detail below. As would be apparent to those of ordinary skill in the art, perforation 209 is not required for sheath 209 to be tearable. For example, as described above, known tearable prior art introducer sheaths may comprise grooves or score lines or axially oriented polymeric material that will promote a longitudinal tear. Further, instead of being tearable, sheath 206 may be made of material sufficiently compliant such that it can stretch radially over a closure device 121, as explained in more detail below.

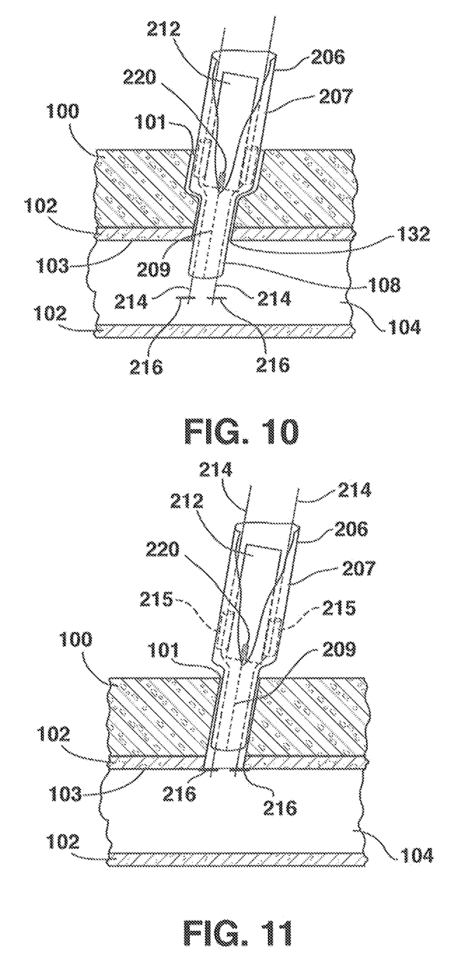

After completion of an intra-luminal procedure and removal of the procedural device, a vascular closure system in accordance with the current embodiment is used to close arteriotomy 132, as follows. As shown in FIG. 9, a guiding system including a pair of stabilization wire guides 214 is inserted into sheath 206, which may be indwelling at the end of the procedure, or inserted anew, or sheath 206 may replace a sheath that was used during the procedure. The guiding system is advanced through sheath 206 and into lumen 104, as shown in FIGS. 9 and 10. A distal portion of each stabilization wire guide 214 includes a retention foot 216. The guiding system is advanced through sheath 206 and into lumen 104 in an undeployed configuration wherein retention feet 216 are unexpanded, undeployed, or hingedly disposed substantially parallel to stabilization wire guides 214, as shown in FIG. 9, to provide a low cross-sectional profile during insertion. In the embodiment shown in FIG. 9, closure device 212 is advanced into sheath 206 together with the guiding system. However, it would be understood by one of ordinary skill in the art that the guiding system may be advanced into sheath 206 without closure device 212.

As shown in FIG. 10, the guiding system is advanced far enough into lumen 104 of vessel 102 to ensure that retention feet 216 are intraluminal prior to their deployment. Numerous methods are known to those of skill in the art to measure or accurately indicate the intraluminal position of portions of a vascular closure device. In one known device, intraluminal position of the device may be indicated by blood entering the device through an inlet port positioned within the vessel lumen, the blood flowing through a passageway in the device, and the blood finally emitting from the device as a visible “flashback” out of an exit port external to the patient. In an alternative known type of position indicator, the device may have an enlarged physical stop for abutting against the external wall of the blood vessel to provide a tactile indication of the position of the device with respect to the vessel wall. In the embodiment shown in FIG. 10, closure device 212 is advanced into sheath 206 together with the guiding system such that closure device 212 causes proximal portion 207 of sheath 206 to tear along perforation 209. As would be understood by one of ordinary skill in the art, an alternative embodiment wherein a proximal portion 207′ of a sheath 206′ stretches over closure device 212 may be utilized, as shown in FIG. 16. Further, closure device 212 need not be advanced with the guiding system. In such an embodiment, proximal portion 207 would not be torn or stretched as the guiding system is advanced into sheath 206 and into lumen 104.

Once retention feet 216 are positioned intraluminally, the guiding system is transformed into a deployed configuration such that retention feet expand or project laterally from wire guides 214 within lumen 104. Retention feet 216 may be formed and deployed in various ways as would be known to those of ordinary skill in the art. For example, several retention feet are described in the above-mentioned Kanner '356 patent. After retention feet 216 have been deployed such that they are generally perpendicular to stabilization wire guides 214, the entire system, including the guiding system, sheath 106, and closure device 212, if already present on the guiding system, is moved proximally until tactile resistance is felt due to retention feet 216 abutting the inner surface of vessel wall 103, as shown in FIG. 11.

The contact of retention feet 216 against the inner surface of vessel wall 103 provides temporary anchoring for stabilization wire guides 214, and also provides an indication of the location, or depth of vessel wall 203 relative to other components of the vascular closure system. For convenience of illustration, FIGS. 9-14 show a pair of stabilization wire guides 214 aligned along the axis of vessel 102. In actual use however, such a pair of stabilization wire guides 214 will typically be aligned transverse to the axis of vessel 102 and positioned at the ends of the slit that naturally forms generally perpendicular to the length of the vessel when arteriotomy 132 is formed in the wall of vessel 102, as described in the above-mentioned Kanner '356 patent. Such transverse alignment of stabilization wire guides 214 within arteriotomy 132 is especially typical when stabilization wire guides 214 are spread apart by closure system 212, as described below.

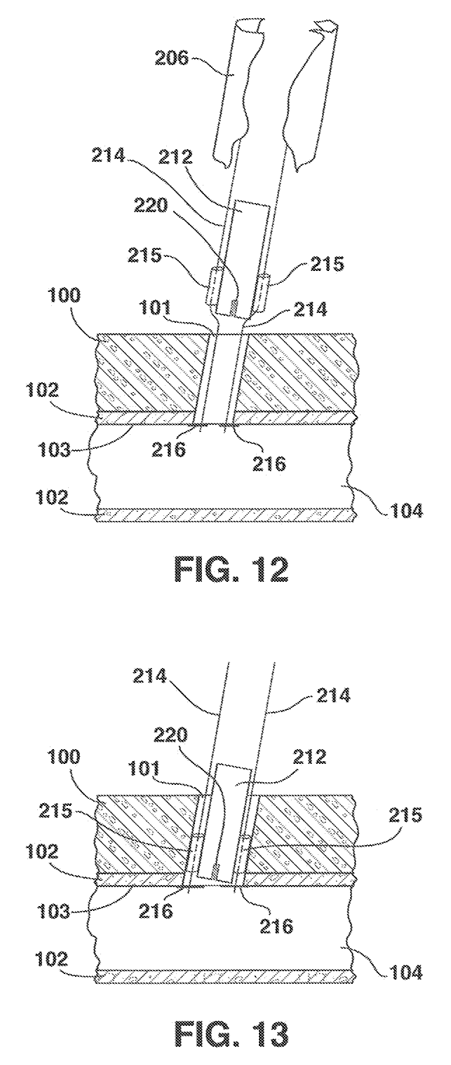

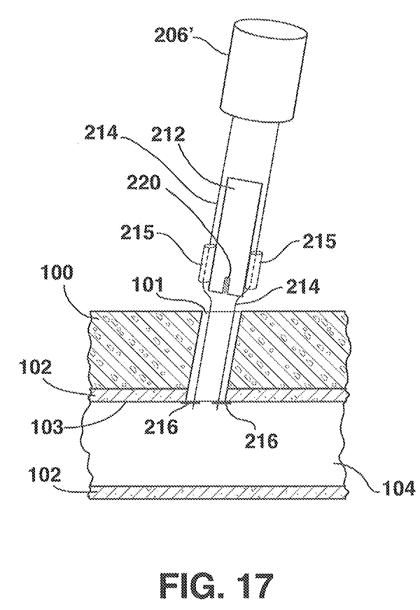

Closure device 212 may be held outside of sheath 206 until after sheath 206 and the guiding system are pulled proximally such that retention feet 216 abut the inner surface of vessel wall 103. In such an embodiment, closure device 212 may then be advanced into proximal portion 207 of sheath 206, thereby tearing proximal portion 207 along perforation 209 or stretching proximal portion 207′ of sheath 206′. With the guiding system temporarily aligned with arteriotomy 132 by stabilization wire guides 214 and anchored by retention feet 216, sheath 206 is pulled back over closure device 212, as shown in FIG. 12. In the embodiment shown in FIG. 12, sheath 206 continues to tear along perforation 209 as sheath 206 is pulled proximally over closure device 212. However, one of ordinary skill in the art would recognize that, instead of tearing, sheath 206′ may be sufficiently pliant that sheath 206′ may stretch radially over closure device 212, as shown in FIG. 17. Further, as described above, proximal portion 207 of sheath 206 was already torn if closure device 212 and the guiding system were advanced together into sheath 206. In another embodiment, sheath 206 may be pulled proximally over closure device 212 when closure device is located proximally of sheath 206.

After sheath 206 is pulled proximally over closure device 212, closure device 212 is further advanced along stabilization wire guides 214 to a position adjacent arteriotomy 132, as shown in FIG. 13. Closure device 212 may include a pair of side tubes 214 that ride along stabilization wire guide 214. As shown in FIG. 12, two or more relatively short side tubes 215 are coupled parallel to a distal portion of closure device 212 on generally opposing sides of thereof for riding along stabilization wire guides 214. For example, but not as a limitation, closure device 212 of this disclosure may comprise center tube 124 disclosed in U.S. application Ser. No. 11/626,567, as incorporated by reference herein above. Stabilization wire guides 214 are also spread apart from each other by closure system 212 as it is advanced towards arteriotomy 132. With stabilization wire guides 214 positioned at the ends of the arteriotomy slit 132 as described above, spreading stabilization wire guides 214 apart causes the list to lengthen without enlarging arteriotomy 132, thus drawing the opposite edges of the slit closer towards apposition and enhancing the chances of achieving a successful wound closure.

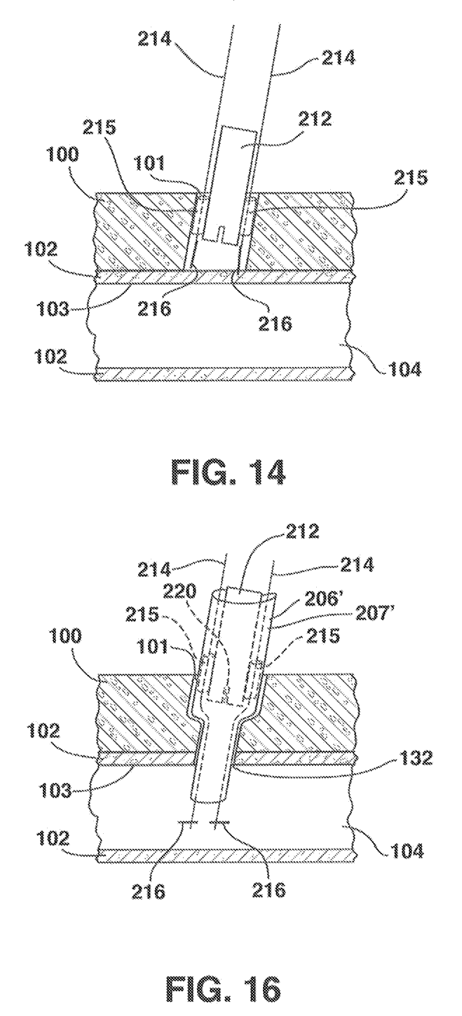

A closure mechanism 220 such as a staple is actuated and/or delivered by closure device 212 to close arteriotomy 132. Any conventional vascular puncture closure device can be used, as would be understood by those or ordinary skill in the art. Non-limiting examples of closure devices include implantable mechanisms such as staples, clips, sutures and plugs, or instruments that weld or bond tissue, as by chemicals, radio frequency or laser energy. In particular, the stapler and staples disclosed in U.S. application Ser. No. 11/626,567 mentioned above may be used to close arteriotomy 132. Several methods for releasing closure mechanism 220 from closure device 212 may be used, such as the methods described in the above-referenced U.S. application Ser. No. 11,626,567. Regardless of the closure device or method used, the opposing edges of arteriotomy 132 are brought into apposition surrounding stabilization wire guides 214. After closure mechanism 220 is actuated and/or delivered, the guiding system is transformed back into the undeployed configuration, such that retention feet 216 are retracted or collapsed into a low cross-sectional profile. Then, the guiding system and closure device 212 are removed from the patient via tissue track 101. FIG. 14 illustrates the guiding system in the undeployed configuration and, along with closure device 212, the guiding system is partially removed from tissue track 101.

While various embodiments of the present disclosure have been described above, it should be understood that they have been presented by way of illustration and example only, and not limitation. It will be apparent to persons skilled in the relevant art that various changes in form and detail can be made therein without departing from the spirit and scope of the disclosure. Thus, the breadth and scope of the present disclosure should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. For example, and not by way of limitation, the method described with respect to FIGS. 1-7, a sheath such as the one described with respect to FIG. 8-14 could be used, or in the method described with respect to FIGS. 8-14, a sheath such as the one described with respect to FIGS. 1-7 could be used. All patents and publications discussed herein are incorporated by reference herein in their entirety.

What is claimed is:

1. A method for closing a wall opening of a vessel of a body, the method comprising the steps of:
providing a guiding system comprising at least two elongate stabilization wire guides and a retention foot disposed at a distal portion of each stabilization wire guide, each retention foot being selectively expandable laterally from the respective stabilization wire guide in at least one direction, the guiding system including a deployed configuration wherein the retention feet are expanded, and an undeployed configuration wherein the retention feet are unexpanded;
providing a closure system including a closure mechanism, wherein the closure system is coupled to at least two opposing side tubes for riding along the stabilization wire guides, respectively;
disposing an access sheath in the opening in the vessel, wherein the sheath includes a distal portion at least partially disposed within the vessel and a proximal portion at least partially disposed outside the body;
delivering the guiding system through the sheath and the wall opening into the vessel in the undeployed configuration;.
delivering the closure system into the sheath to a position a predetermined distance from the opening in the vessel;
transforming the guiding system into the deployed configuration such that the retention feet are expanded within the vessel;
pulling the sheath, guiding system, and closure system proximally to a position such that the retention feet contact an inner wall of the vessel and the distal portion of the sheath is entirely outside of the vessel;
after the pulling step, advancing the closure system along the guiding system through the sheath to a position proximal to the wall opening; and
activating the closure mechanism to close the opening.

2. The method of claim 1, wherein the step of advancing the closure system along the guiding system through the sheath includes tearing the distal portion of the sheath to expose the closure system.

3. The method of claim 2, wherein the sheath is perforated.

4. The method of claim 1, wherein the step of advancing the closure system along the guiding system through the sheath includes stretching the sheath over the closure system to expose the closure system.

5. The method of claim 1, wherein the closure mechanism is a stapler.

6. The method of claim 5, wherein activating the stapler comprises the steps of:
implanting a staple across the opening in the wall of the vessel; and
releasing the staple from the stapler.

7. The method of claim 1, further comprising the step of transforming the guiding system into the undeployed configuration and withdrawing the guiding system from the body after activating the closure mechanism to close the opening.

8. The method of claim 1, wherein the sheath includes a proximal portion having a first diameter and a distal portion have a second diameter, wherein the first diameter is larger than the second diameter.

9. A method for closing a wall opening of a vessel of a body, the method comprising the steps of:
providing a guiding system comprising at least two elongate stabilization wire guides and a retention foot disposed at a distal portion of each stabilization wire guide, each retention foot being selectively expandable laterally from the respective stabilization wire guide in at least one direction, the guiding system including a deployed configuration wherein the retention feet are expanded, and an undeployed configuration wherein the retention feet are unexpanded;
providing a closure system including a closure mechanism, wherein the closure system is coupled to at least two opposing side tubes for riding along the stabilization wire guides, respectively;
providing an access sheath in the opening in the vessel, wherein the sheath includes a distal portion at least partially disposed within the vessel and a proximal portion at least partially disposed outside the body;
delivering the guiding system through the sheath and the wall opening into the vessel in the undeployed configuration;
delivering the closure system into the sheath to a position a predetermined distance from the opening in the vessel;
transforming the guiding system into the deployed configuration such that the retention feet are expanded within the vessel;
pulling the sheath, guiding system, and closure system proximally to a position such that the retention feet contact an inner wall of the vessel and the distal portion of the sheath is entirely outside of the vessel;
after the pulling step, further pulling the sheath proximally over the closure system such that at least a portion of the closure system is disposed distal of the sheath;
after pulling the sheath proximally, further advancing the closure system along the guiding system to a position proximal to the wall opening; and
activating the closure mechanism to close the opening.

10. The method of claim 9, wherein the step of pulling the sheath proximally over the closure system includes tearing the distal portion of the sheath as the sheath is being pulled distally.

11. The method of claim 10, wherein the sheath is perforated.

12. The method of claim 9, wherein the step of pulling the sheath proximally over the closure system includes stretching the sheath over the closure system.

13. The method of claim 9, wherein the closure mechanism is a stapler.

14. The method of claim 13, wherein activating the stapler comprises the steps of:
implanting a staple across the opening in the wall of the vessel; and
releasing the staple from the stapler.

15. The method of claim 9, further comprising the step of transforming the guiding system into the undeployed configuration and withdrawing the guiding system from the body after activating the closure mechanism to close the opening.

16. The method of claim 9, wherein the sheath includes a proximal portion having a first diameter and a distal portion have a second diameter, wherein the first diameter is larger than the second diameter.
